(19) United States
(12) Patent Application Publication
(10) Pub. No.: US 2008/0031891 A1
(43) Pub. Date: Feb. 7, 2008

(54) COMPOSITIONS AND METHODS FOR REDUCING OR PREVENTING PAIN IN A SUBJECT

(76) Inventor: Ronald Lewis, Acworth, GA (US)

Correspondence Address: GARDNER GROFF GREENWALD & VILLANUEVA. PC, 2018 POWERS FERRY ROAD, SUITE 800, ATLANTA, GA, 30339, US

(21) Appl. No.: 11/613,465

(22) Filed: Dec. 20, 2006

Related U.S. Application Data
(60) Provisional application No. 60/821,600, filed on Aug. 7, 2006.

(51) Int. Cl. A61K 36/06; A61K 36/539; A61K 36/61; A61K 36/53; A61K 36/534; A61K 36/906; A61K 36/48

(52) U.S. Cl. 424/195.15; 424/769; 424/747; 424/756; 424/757; 424/745; 424/741; 424/742

(57) ABSTRACT

Described herein are compositions derived from natural products useful in reducing or preventing pain in a subject and methods of use thereof.

### IN THE CROSS-REFERENCE TO RELATED APPLICATIONS

This application claims priority to U.S. Provisional Application Ser. No. 60/821,600, filed Aug. 7, 2006, which is hereby incorporated herein by reference in its entirety for all purposes.

### BACKGROUND

Pain is general phenomena that has many sources and inflicts millions of people on a daily basis. The source of pain can arise from injury or disease to name just a few. The level or degree of pain can vary from very mild to extremely debilitating. In the case of extreme pain, not only is the subject exposed to significant discomfort, the quality of life of loved ones can also be adversely affected, particularly if they have to see a family member experiencing pain over prolonged periods of time.

One source of pain is derived from peripheral neuropathy. As many as 20 million people in the U.S. suffer from peripheral neuropathy. Peripheral neuropathy is the medical term for damage to nerves of the peripheral nervous system, which may be caused either by diseases of the nerve or from the side-effects of systemic illness. Peripheral neuropathies vary in their presentation and origin, and may affect the nerve or the neuromuscular junction. Major causes of peripheral neuropathy include seizures, nutritional deficiencies, alcoholism and HIV, though diabetes is the most likely cause. Mechanical pressure from staying in one position for too long, a tumor, intraneural hemorrhage, exposing the body to extreme conditions such as radiation, cold temperatures, or toxic substances (including some chemotherapy drugs such as vincristine) can also cause peripheral neuropathy.

When peripheral neuropathy first develops, people often report a tingling or prickling in the toes, although it can also start in the fingers. Over time, the tingling gradually spreads up the feet or hands and worsens into a burning, shooting, and/or throbbing pain. Thus, peripheral neuropathy almost always occurs in both feet and/or both hands. Symptoms generally occur on both sides of the body. Symptoms can vary in degree from barely noticeable to very debilitating. The symptoms can be either constant or periodic.

There are numerous medications that are available for reducing pain. Examples include pain relievers (e.g., acetaminophen and non-steroidal anti-inflammatory drugs), anti-seizure medications (e.g., gabapentin, carbamazepine, and phenytoin), lidocaine patches, tricyclic antidepressants (e.g., amitriptyline, nortriptyline), and analgesics. However, these medications have undesirable side effects. For example, pain relievers can cause nausea, stomach pain, bleeding, or ulcers. Therapies such as transcutaneous electrical nerve stimulation, biofeedback, acupuncture, hypnosis, and relation techniques can be used alone or in combination with medication to relieve pain. However, these techniques may not work for everyone.

Thus, new treatments for reducing or preventing pain in a subject are needed that do not have undesirable side effects. It is preferred that the treatment be derived from natural sources and easily administered to the subject in need of such treatment.

### SUMMARY

Described herein are compositions derived from natural products useful in reducing or preventing pain in a subject and methods of use thereof. The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not.

By “subject” is meant an individual. The subject can be a mammal such as a primate or a human. The term “subject” can include domesticated animals including, but not limited to, cats, dogs, etc., livestock (e.g., cattle, horses, pigs, sheep, goats, etc.), and laboratory animals (e.g., mouse, rabbit, rat, guinea pig, etc.).

By “contacting” is meant an instance of exposure by close physical contact of at least one substance to another substance. For example, contacting can include contacting a substance, such as a pharmacologic agent, with a cell. The duration of contact with a cell or group of cells is determined by the time the test compound is present at physiologically effective levels or at presumed physiologically effective levels in the medium or extracellular fluid bathing the cell.

“Treatment” or “treating” means to administer a composition to a subject or a system with an undesired condition (e.g., pain) or at risk for the condition. The condition can include a disease or a predisposition to a disease. The effect of the administration of the composition to the subject can have the effect of but is not limited to reducing or preventing the symptoms of the condition, a reduction in the severity of the condition, or the complete ablation of the condition.

By “effective amount” is meant a therapeutic amount needed to achieve the desired result or results, e.g., reduce or prevent pain.

Described herein are compositions derived from natural products useful in reducing or preventing pain in a subject and methods of use thereof. The compositions generally contain one or more oils or extracts from natural products possessing particular properties. In one aspect, the oils and extracts used to produce the compositions described herein possess antisickling activity, antiinflammatory activity, antiprostaglandin activity, and vasoconstrictor/vasodilator activity.

The term “antisickling” is defined herein as activites used to prevent or reverse the pathological events leading to sickling of erythrocytes in sickle cell conditions. Sources of antisticking compounds can be derived from the non-limiting list of natural products:

Lupinus albus (White Lupine) Seed

Helianthus annuus (Girasol) Seed

Nasturtium officinale (Berro) Herb

Nigella sativa (Black Cumin) Seed

Glycyrrhiza glabra (Licorice) Root

Ceratonia siliqua (Carob) Seed

Glycine max (Soybean) Seed

Phaseolus vulgaris subsp. var. vulgaris (Haricot) Sprout Seedling

Cucurbita foetidissima (Buffalo Gourd) Seed

Arachis hypogaea (Peanut) Seed

Senna obtusifolia (Sicklepod) Seed

Corchorus olitorius (Mulukiya) Leaf

Cucurbita pepo (Pumpkin) Seed

Amaranthus sp. (Pigweed) Leaf

Spinacia oleracea (Spinach) Plant

Ipomoea aquatica (Swamp Cabbage) Leaf

Juglans cinerea (Butternut) Seed

Symphytum officinale (Comfrey) Root

Vanilla planifolia (Vanilla) Fruit

Sesamum indicumi (Beni) Seed

Foeniculum vulgare (Fennel) Fruit

Pisum sativum (Pea) Seed

Triticum aestivum (Wheat) Seed

Allium cepa (Onion) Bulb

Ananas comosus (Pineapple) Fruit

Avena sativa (Oats) Seed

Glycine max (Soybean) Seed

Theobroma cacao (Cacao) Seed

Arachis hypogaea (Peanut) Seed

Asparagus officinalis (Asparagus) Shoot

Beta vulgaris subsp. subsp. vulgaris (Beet) Root

Capsicum annuum (Paprika) Fruit

Capsicum frutescens (Chili) Fruit

Citrus sinensis (Orange) Fruit

Daucus carota (Carrot) Root

Helianthus annuus (Girasol) Seed

Lupinus albus (White Lupine) Seed

Panax quinquefolius (Ginseng) Plant

Phoenix dactylifera (Date Palm) Fruit

Prunus cerasus (Sour Cherry) Fruit

Salvia officinalis (Sage) Plant

The term “antiinflammatory” is defined herein as any oil or extract that reduces or prevent inflammation in a subject. Sources of antiinflammatory compounds can be derived from natural products such as, for example:

Persea americana (Avocado) Fruit

Macadamia spp (Macadamia) Seed

Cucumis melo subsp. ssp melo var. cantalupensis (Melon) Cotyledon

Prunus armeniaca (Apricot) Seed

Citrus limon (Lemon) Pericarp Essent. Oil

Bertholletia excelsa (Paranut) Seed

Pistacia vera (Pistachio) Seed

Thymus vulgaris (Thyme) Plant

Anacardium occidentale (Cashew) Seed

Juglans cinerea (Butternut) Seed

Origanum vulgare (Oregano) Plant

Foeniculum vulgare (Fennel) Fruit

Daucus carota (Carrot) Root

Ribes nigrum (Black Currant) Fruit

Rosmarinus officinalis (Rosemary) Plant

Camellia sinensis (Tea) Leaf

Citrus paradisi (Grapefruit) Fruit

Matricaria recutita (Wild Camomile) Plant

Lycopersicon esculentum (Tomato) Fruit

Coriandrum sativum (Cilantro) Fruit

Capsicum annuum (Paprika) Fruit

Glycyrrhiza glabra (Licorice) Root

Achillea millefolium (Yarrow) Plant

Allium cepa (Onion) Bulb

Capsicum frutescens (Chili) Fruit

Glycine max (Soybean) Seed

Vaccinium corymbosum (Blueberry) Plant

Chamaemelum nobile (Roman Camomile) Plant

Panax quinquefolius (Ginseng) Plant

Vitis vinifera (Grape) Fruit

The term “antiprostaglandin” is defined herein as any oil or extract that reduces the release of prostaglandins, which are a group of potent hormonelike substances that are produced in various mammalian tissues derived from arachidonic acid. Prostaglandins mediate a wide range of physiological functions, such as control of blood pressure, contraction of smooth muscle, modulation of inflammation that can cause swelling and pain. Sources of antiprostaglandin compounds can be derived from natural products such as, for example:

Syzygium aromaticum (Clove) Flower

Glycyrrhiza glabra (Licorice) Root

Abrus precatorius (Jequerity) Leaf

Vitis vinifera (Grape) Leaf

Origanum vulgare (Oregano) Plant

Pimenta dioica (Pimenta) Fruit

Citrus limon (Lemon) Pericarp

Hyssopus officinalis (Hyssop) Plant

Citrus sinensis (Orange) Pericarp

Prunella vulgaris (Heal-All) Plant

Thymus vulgaris (Thyme) Plant

Mentha spicata (Spearmint) Plant

Origanum majorana (Marjoram) Plant

Ocimum basilicum (Basil) Leaf

Conium maculatum (Hemlock) Plant

Ipomoea purga (Jalap) Tuber

Rosmarinus officinalis (Rosemary) Tissue Culture

Curcuma longa (Turmeric) Rhizome

Pimenta racemosa (Bayrum Tree) Leaf

Piper betel (Betel Pepper) Leaf

Hyssopus officinalis (Hyssop) Plant

Origanum majorana (Marjoram) Plant

Vaccinium corymbosum (Blueberry) Plant

Allium cepa (Onion) Bulb

Allium sativum var. sativum (Garlic) Bulb

Angelica archangelica (Angelica) Plant

Capsicum frutescens (Chili) Fruit

Coriandrum sativum (Cilantro) Fruit

Daucus carota (Carrot) Root

Lycopersicon esculentum (Tomato) Fruit

Mentha pulegium (European Pennyroyal) Plant

The term “vasodilator” is defined herein as any oil or extract that widens blood vessels, respectively in a subject. Pain has been associated with the narrowing of blood vessels. Sources of vasodilators can be derived from natural products such as, for example:

Mentha spicata (Spearmint) LeafCarthamus tinctorius (Safflower) Plant

Opuntia ficus-indica (Nopal) Seed

Avena sativa (Oats) Plant

Allium ampeloprasum (Kurrat) Plant

Angelica sinensis (Dang Gui) Root

Capsicum annuum (Paprika) Fruit

Foeniculum vulgare (Fennel) Fruit

Glycyrrhiza glabra (Licorice) Root

Oenothera biennis (Evening-Primrose) Seed

Tetrapanax papyrifera (Tong-Cao) Pith

Hordeum vulgare (Barley) Plant

Linum usitatissimum (Flax) Hay

Canavalia ensiformis (Jack Bean) Fruit

Triticum aestivum (Wheat) Plant

Medicago sativa subsp. sativa (Alfalfa) Plant

Pueraria montana subsp. var. lobata (Kudsu) Stem

Psidiu guajava (Guava) Fruit

Helianthus annuus (Girasol) Seed

Solanum torvum (Susumba) Fruit

Vetiveria zizanioides (Cus-Cus) Leaf

Capsicum annuum (Paprika) Fruit

Psidium guajava (Guava) Seed

Panax quinquefolius (Ginseng) Plant

Camellia sinensis (Tea) Leaf

Lycopersicon esculentum (Tomato) Fruit

Musa x paradisiaca (Banana) Fruit

Capsicum annuum (Paprika) Fruit

Foeniculum vulgare (Fennel) Fruit

Ribes nigrum (Black Currant) Fruit

Origanum vulgare (Oregano) Plant

Achillea millefolium (Yarrow) Plant

Brassica oleracea var. botrytis l. var. botrytis (Cauliflower) Leaf

Capsicum frutescens (Chili) Fruit

Citrus paradisi (Grapefruit) Fruit

Ocimum basilicum (Basil) Leaf

Vitis vinifera (Grape) Fruit

Allium cepa (Onion) Bulb

Allium sativum var. sativum (Garlic) Bulb

Allium schoenoprasum (Chives) Leaf

Asparagus officinalis (Asparagus) Shoot

Brassica oleracea var. capitata l. var. capitata (Cabbage) Leaf

Brassica oleracea var. sabellica l. var. acephala (Kale) Leaf

Glycine max (Soybean) Seed

In one aspect, the composition comprises an extract of one or more mushrooms. Mushroom extracts are known in the art, and include extracts from, wherein the mushroom extract comprises an extract from Shiitake mushroom, Maitake mushroom, Cordyceps mushroom, Turkey Tail mushroom, Tremella mushroom, Lion's Mane mushroom, Agaricus mushroom, Button mushroom, or any combination thereof. Mushroom extracts commercially available from a number of different manufacturers can be used herein. In one aspect, an extract from Shiitake mushrooms can be used. Not wishing to be bound by theory, Shiitake extracts show an inhibitory effect on water-insoluble glucan formation from sucrose by crude glucosyltransferases of Streptococcus mutans JC-2 and Streptococcus sobrinus OMZ-176. Shiitake extracts also possess antioxidant activity, immunopotentiating properties, and other protective qualities within body cells. Shiitake extract manufactured by Natures Answer can be used herein.

In another aspect, the composition comprises one or more fig extracts, Figs are antibacterial, antiparasitic, and restorative materials. Figs contain the compound, benzaldehyde, which according to Japanese studies, shrink tumors. Figs also contain a sulphur compound called ficin, which helps reduce joint and tissue inflammation and is used to treat injuries. Figs are used to treat anemia, arthritis, asthma, low blood pressure, cancer, catarrh, colitis, coughs, dysentery, emaciation, exhaustion, gout, hemorrhoids, pleurisy, rheumatism, sore throat, tuberculosis, and ulcers. Figs are rich in B6, folic acid, calcium, copper, iron, magnesium, manganese, phosphorus, and potassium. Fig extracts manufactured by Rachel's Supply can be used herein.

In one aspect, the composition comprises a mixture of mushroom and fig extracts. The fig extract extends pain relief to the subject when used in combination with the mushroom extract. The amount of fig extract relative to the amount of mushroom extract can vary. In one aspect, equal amounts by weight or volume of fig and mushroom extract can be used in this aspect.

It is contemplated that additional components can be added to the compositions described herein that possess other desirable activities. Examples of such components include, but are not limited to, an aldose-reductase inhibitor, a beta-blocker, an antioxidant, an analgesic, an angiotensin-receptor blocker, or an antileukotriene.

In one aspect, the composition comprises an extract or oil from comfrey, witch hazel, licorice, elder, calendula, shiitake mushroom, and fig.

Comfrey (Symphytum officinale L.) is a perennial herb of the family Boraginaceae. Comfrey has numerous properties including antioxidant activity, antisickling activity, angiotensin-receptor blocker activity, antihypertensive activity, antiinflammatory activity, and beta-blocker activity. In one aspect, extracts derived from fresh roots of comfrey are useful. Comfrey extract manufactured by Herb Pharm can be used herein.

Witch hazel is the common name for a genus of shrubs Hamamelis in the family Hamamelidaceae. The main constituents of the extract include tannin, gallic acid, catechins, proanthocyanins, flavonoids (kaempferol, quercitin), essential oil (carvacrol, eugenol, hexenol), choline, saponins, and bitters. Witch hazel has numerous properties including antioxidant activity, antiinflammatory activity, analgesic activity, and myorelaxant activity. In one aspect, extracts derived from leaves and twigs of witch hazel are useful. Witch hazel extract manufactured by Herb Pharm can be used herein.

Licorice is the root of Glycyrrhiza glabra. Licorice has numerous properties including antisickling activity, antiinflammatory activity, beta-blocker activity, a MAO-inhibitor, a prostaglandin synthesis inhibitor, and a vasodilator. In one aspect, extracts derived from roots and stolons are useful. Licorice extract manufactured by Herb Pharm can be used herein.

Elder or Elderberry (Sambucus) is a genus of between 5-30 species of fast-growing shrubs or small trees (two species herbaceous) in the moschatel family Adoxaceae. Elder has numerous activities including an aldose-reductase-inhibitor, an analgesic, an antihistaminic, an antileukotriene, an antihypertensive, an antiinflammatory, an antileukotriene, an antioxidant, a histamine inhibitor, a leukotriene inhibitor, a hypotensive, a topoisomerase-II inhibitor, and a vasodilator. In one aspect, extracts derived from flowering umbels of elder are useful. Elder extract manufactured by Herb Pharm can be used herein.

Calendula (marigold) is a genus of about 12-20 species of annual or perennial herbaceous plants in the daisy family Asteraceae. Calendula has several desirable properties including, but not limited to, an aldose-reductase inhibitor, an analgesic, an angiotensin-receptor blocker, an antihistaminic, an antihypertensive, an antiinflammatory, an antioxidant, a beta-adrenergic receptor blocker, a hypotensive, and a vasodilator. Calendula extract manufactured by Herb Pharm can be used herein.

In another aspect, the composition comprises an extract or oil from comfrey, witch hazel, licorice, elder, calendula, shiitake mushroom, fig, skullcap, rosemary, Hawthorne blend, and ginger. In a further aspect, this composition further comprises an oil or extract from witch hazel.

Scutellaria is a genus of about 300 species of plants commonly known as skullcaps. Skullcaps have the following non-limiting propereties: an aldose-reductase inhibitor, an analgesic, an angiotensin-receptor blocker, an antihistaminic, an antihypertensive, an antiinflammatory, an antioxidant, a beta-adrenergic receptor blocker, a beta-blocker, a calcium-channel blocker, a hypotensive, an immunostimulant, an insulinogenic, and a vasodilator. In one aspect, extracts derived from fresh flowering herbs of skullcaps are useful. Skullcap extracts manufactured by Herb Pharm can be used herein.

Rosemary (Rosmarinus officinalis) is a woody, perennial herb with fragrant evergreen needle-like leaves. Rosemary has the following non-limiting propereties: an alpha-reductase inhibitor, an angiotensin-receptor blocker, an antidiabetic, an antihypertensive, an antihistaminic, an antiinflammatory, an antieukotriene, an antioxidant, an antisickling, a beta-B calcium-antagonist locker, a hypotensive, lipoxygenase inhibitor, and a neuroinhibitor. In one aspect, extracts derived from fresh flowering branches of rosemary are useful. Rosemary extracts manufactured by Herb Pharm can be used herein.

Crataegus (Hawthorn) is a large genus of shrubs and small trees in the family Rosaceae. Useful species of hawthorn include crataegus monogyna and oxyacantha. Hawthorn has the following non-limiting propereties: an aldose-reductase inhibitor, an analgesic, an antiinflammatory, an antioxidant, a hypotensive, an antioxidant, an antiprogestational, and an antiprostaglandin. In one aspect, extracts derived from flowers, leaves, and berries of hawthorn are useful. Hawthorn extracts manufactured by Herb Pharm can be used herein.

Ginger belongs to the family Zingiberaceae. Ginger has the following non-limiting properties: an aldose-reductase inhibitor, an analgesic Anti-5-HT, an antihistaminic, an antiinflammatory, an antiprostaglandin, an antioxidant, a hypotensive, and a prostaglandin-synthesis inhibitor. In one aspect, extracts derived from mature rhizomes of ginger are useful. Ginger extracts manufactured by Herb Pharm can be used herein.

The compositions described herein can include other components. In one aspect, the composition further comprises an absorption enhancer. Examples of absorption enhancers include, but are not limited to, peppermint spirits, eucalyptus oil, emu oil from the emu bird, or a combination thereof. The amount of absorption enhancer can vary depending upon the selection of components used to prepare the composition. In one aspect, the amount of absorption enhancer is one-half part by volume relative to the other components.

In another aspect, the compositions described herein further comprise mucilage. Mucilage is an exopolysaccharide, which is a thick gluey substance produced by most plants. In the compositions described herein, mucilage is used to soothe, coat, and protect inflamed tissue. In one aspect, the source of mucilage can be derived from a mixture of licorice (described above) and marshmallow. Marshmallow has the following non-limiting properties: an angiotensin-receptor blocker, an antiinflammatory, an antisickling, a beta-blocker, a hypotensive, a MAO-inhibitor, and a vasodilator. Extracts from the white mallow root of the marshmallow plant (Althaea officinalis) can be used as the marshmallow component. In one aspect, ½ part by volume marshmallow and ½ part by volume licorice is used to provide a higher mucilage content.

The compositions described herein can be manufactured using techniques known in the art. In one aspect, the extracts or oils can be admixed with one another by shaking or stirring the mixture. The selection and amounts of each component can vary depending upon the type and source of pain. Additionally, the selection of components can be based upon compatibility with the subject. For example, the subject may be allergic to certain components. In this situation, it is possible to substitute or replace components to avoid undesirable side effects. In one aspect, the composition comprises an extract or oil from comfrey, witch hazel, licorice, elder, calendula, shiitake mushroom, and fig, where there is one part by volume of each component. In another aspect, the composition comprises an extract or oil from comfrey, witch hazel, licorice, elder, calendula, shiitake mushroom, fig, skullcap, rosemary, Hawthorne blend, and ginger, where there is one part by volume of each component.

Although the compositions described herein can be administered topically to a subject as an aqueous solution or oil, it is contemplated that the compositions can be formulated in ointments, lotions, creams, gels, drops, sprays, liquids and powders. Conventional pharmaceutical carriers, aqueous, powder or oily bases, thickeners and the like can be used as necessary.

The compositions described herein are useful in reducing or preventing pain in a subject. The source of pain can be derived from numerous sources. In one aspect, the source of pain is caused by peripheral neuropathy. Symptoms of peripheral neuropathy include sharp, jabbing pain to the feet and hands. Other forms of pain include a burning sensation and extreme sensitivity to touch. There are many types of peripheral neuropathies. Distal axonopathies are caused by metabolic diseases such as diabetes, renal failure, deficiency syndromes such as malnutrition and alcoholism, or the effects of toxins or drugs. Myelinopathies are due to the attack of myelin. Guillain-Barré syndrome, chronic inflammatory demyelinating syndrome (CIDP), and genetic metabolic disorders (e.g., leukodystrophy), or toxins can cause myelinopathies. Neuronopathies are the result of destruction of peripheral nervous system (PNS) neurons. They can be caused by motor neurone diseases, sensory neuronopathies (e.g., Herpes zoster), toxins or autonomic dysfunction. Neurotoxins can also cause neuronopathies, such as the chemotherapy agent vincristine. In another aspect, pain derived from idiopathic peripheral neuropathy, hereditary neuropathy with liability to pressure palsies (HNPP), inflamatory/systemic compression or sickle cell anemia can be reduced or prevented using the compostions described herein. In other aspects, the described herein can be used to treat or prevent pain from joint disorders such as, for example, arthritis. Not wishing to be bound by theory, it is believed that the compositions described herein either directly or indirectly reduce or prevent the uptake or release of electrolytes (e.g., sodium or calcium) or free from cells, which are associated with pain and inflammation.

The compositions described herein are generally applied directly to the source of the pain in the subject. In one aspect, the compositions are applied topically to the skin. The composition can be massaged into the skin in order to ensure that the skin absorbs the composition. The compositions can be applied to the skin as needed. Dosing is dependent on severity and responsiveness of the condition to be treated, but will normally be one or more doses per day, with course of treatment lasting from several days to several months or until one of ordinary skill in the art determines the delivery should cease. Persons of ordinary skill can easily determine optimum dosages, dosing methodologies and repetition rates. The duration of pain relief can vary depending upon several factors including the severity of the condition, physical stress exerted on the body, and the temperature of the surroundings. In one aspect, the subject can experience pain relief within 1 minute to 30 minutes after administration for a period of from 6 hours to 72 hours.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

What is claimed:

1. A method for reducing or preventing pain in a subject comprising topically administering to the subject in need of such treatment a composition comprising one or more mushroom extracts, one or more fig extracts, or a combination thereof.

2. The method of claim 1, wherein the administration comprises contacting inflamed tissue with the composition.

3. The method of claim 1, wherein the pain is caused by peripheral neuropathy.

4. The method of claim 1, wherein the pain is caused by arthritis.

5. The method of claim 1, wherein the mushroom extract comprises an extract from Shiitake mushroom, Maitake mushroom, Cordyceps mushroom, Turkey Tail mushroom, Tremella mushroom, Lion's Mane mushroom, Agaricus mushroom, Button mushroom, or any combination thereof.

6. The method of claim 1, wherein the composition further comprises an extract of comfrey, witch hazel, licorice, elder, calendula, skullcap, rosemary, Hawthorne blend, ginger, or any combination thereof.

7. The method of claim 1, wherein the composition further comprises an absorption enhancer.

8. The method of claim 7, wherein absorption enhancer comprises peppermint spirits, eucalyptus oil, or a combination thereof.

9. The method of claim 1, wherein the composition further comprises mucilage.

10. The method of claim 1, wherein the composition further comprises an extract or oil that is an aldose-reductase inhibitor activity, a beta-blocker, an antioxidant, an analgesic, an angiotensin-receptor blocker, or an antileukotriene.

11. A topical composition for reducing or preventing pain in a subject comprising one or more mushroom extracts and a fig extract.
